(19) United States
(12) Patent Application Publication
(10) Pub. No.: US 2026/0369418 A1
(43) Pub. Date: Nov. 19, 2026

(54) HEALTH CONDITION EVALUATION SYSTEM, HEALTH CONDITION EVALUATION METHOD, AND NON-TRANSITORY COMPUTER-READABLE MEDIUM

(71) Applicant: NEC Corporation, Tokyo (JP)

(72) Inventors: Osamu HISAMATSU, Tokyo (JP); Mitsuru Noma, Tokyo (JP); Keisuke Suzuki, Tokyo (JP)

(73) Assignee: NEC Corporation, Tokyo (JP)

(21) Appl. No.: 19/657,264

(22) Filed: Apr. 24, 2026

(30) Foreign Application Priority Data
May 15, 2025 (JP) 2025-081903

(51) Int. Cl. G16H 50/30; A63B 24/00; A63B 71/06; G16H 40/67; G16H 50/20

(52) U.S. Cl. CPC G16H 50/30; A63B 24/0006; A63B 71/0622; G16H 40/67; G16H 50/20; A63B 2220/72; A63B 2230/00

(57) ABSTRACT

A health condition evaluation system according to the present disclosure includes at least one memory storing instructions, and at least one processor configured to execute the instructions to acquire biological data of a user, acquire motion data indicating a motion of the user, acquire environment data indicating an environment in which the user is placed, and evaluate a health condition of the user based on the biological data, the motion data, and the environment data. This makes it possible to appropriately support the decision making regarding the health condition of the user.

### INCORPORATION BY REFERENCE

This application is based upon and claims the benefit of priority from Japanese patent application No. 2025-081903, filed on May 15, 2025, the disclosure of which is incorporated herein in its entirety by reference.

### TECHNICAL FIELD

The present disclosure relates to a health condition evaluation system, a health condition evaluation method, and a non-transitory computer readable medium.

### BACKGROUND ART

Japanese Unexamined Patent Application Publication No.H10-302188 discloses a technology for diagnosing health of a user. A remote health care terminal according to Japanese Unexamined Patent Application Publication No.H10-302188 acquires a health information signal of a user from various sensors. The remote health care terminal acquires an activity information signal regarding an activity state of an individual. The remote health care terminal diagnoses health of the user by correcting a health area pattern for the health information signal of the user based on the activity information signal.

### SUMMARY

The health condition of the user can be affected not only by the activity state of the user but also by various factors. In Japanese Unexamined Patent Application Publication No.H10-302188, the health of the user is merely diagnosed based on the activity information signal of the user. Therefore, in Japanese Unexamined Patent Application Publication No.H10-302188, there is a possibility that the health condition of the user cannot be appropriately evaluated.

The present disclosure has been made to solve such a problem, and an example object thereof is to provide a health condition evaluation system, a health condition evaluation method, and a non-transitory computer-readable medium capable of appropriately evaluating a health condition of a user.

A health condition evaluation system according to an example aspect of the present disclosure includes at least one memory storing instructions, and at least one processor configured to execute the instructions to acquire biological data of a user, acquire motion data indicating a motion of the user, acquire environment data indicating an environment in which the user is placed, and evaluate a health condition of the user based on the biological data, the motion data, and the environment data.

In a health condition evaluation method according to an example aspect of the present disclosure, a computer is caused to execute acquiring biological data of a user, acquiring motion data indicating a motion of the user, acquiring environment data indicating an environment in which the user is placed, and evaluating a health condition of the user based on the biological data, the motion data, and the environment data.

A non-transitory computer-readable medium according to an example aspect of the present disclosure stores a program for causing a computer to execute a step of acquiring biological data of a user, a step of acquiring motion data indicating a motion of the user, a step of acquiring environment data indicating an environment in which the user is placed, and a step of evaluating a health condition of the user based on the biological data, the motion data, and the environment data.

According to the present disclosure, it is possible to provide a health condition evaluation system, a health condition evaluation method, and a non-transitory computer readable medium capable of appropriately evaluating a health condition of a user.

### BRIEF DESCRIPTION OF DRAWINGS

The above and other aspects, features, and advantages of the present disclosure will become more apparent from the following description of certain example embodiments, taken in conjunction with the accompanying drawings, in which:

FIG. 1 is a block diagram illustrating a configuration of a health condition evaluation system according to the present disclosure;

FIG. 2 is a flowchart illustrating an example of a flow of a health condition evaluation method by the health condition evaluation system;

FIG. 3 is a block diagram illustrating a configuration of another health condition evaluation system according to the present disclosure;

FIG. 4 is a block diagram illustrating a configuration of an evaluation apparatus;

FIG. 5 is a block diagram illustrating contents stored in a storage unit;

FIG. 6 is a flowchart illustrating an example of a health condition evaluation method by the health condition evaluation system;

FIG. 7 is a flowchart illustrating another example of a health condition evaluation method by the health condition evaluation system; and

FIG. 8 is a diagram illustrating a hardware configuration example of the health condition evaluation system.

### EXAMPLE EMBODIMENT

### First Example Embodiment

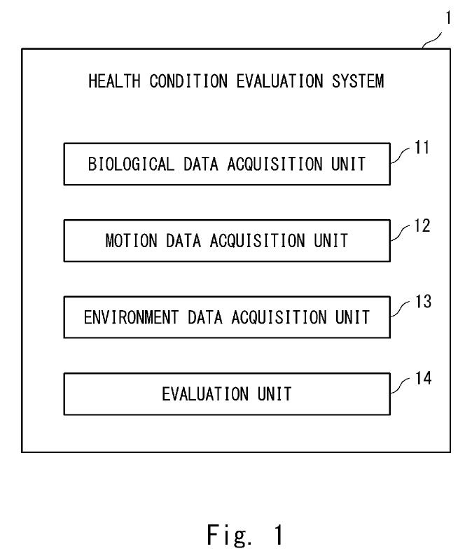

A first example embodiment according to the present disclosure will be described below with reference to the drawings. FIG. 1 is a block diagram illustrating a configuration of a health condition evaluation system 1 according to the present disclosure. The health condition evaluation system 1 is typically one or more computer apparatuses that operate in a case where a processor executes a program stored in a memory. In other words, the health condition evaluation system 1 may include one computer apparatus or two or more computer apparatuses. For example, the health condition evaluation system 1 is one or more server apparatuses.

The health condition evaluation system 1 is an information processing system that evaluates a health condition of a user. The “user” may or may not be a patient at a given medical institution. The user may be a healthy person. The “health condition” refers to determining how healthy the user is. The health condition typically refers to physical health, but may also refer to mental health.

The health condition evaluation system 1 includes a biological data acquisition unit 11, a motion data acquisition unit 12, an environment data acquisition unit 13, and an evaluation unit 14. The elements of the health condition evaluation system 1 typically include software or modules in which processing is executed by the processor executing a program stored in the memory. These elements may be hardware such as a circuitry or a chip. These elements may be configured by the same computer apparatus or may be configured by different computer apparatuses.

The biological data acquisition unit 11, the motion data acquisition unit 12, the environment data acquisition unit 13, and the evaluation unit 14 are data-communicably connected. Data communication between each data acquisition unit and the evaluation unit 14 may be performed via a wired or wireless network. The data communication may be performed in the same computer apparatus, may be performed via an Internet line, or may be performed by using a near field communication technology. A type of a communication protocol in data communication is not limited. The biological data acquisition unit 11, the motion data acquisition unit 12, and the environment data acquisition unit 13 may be connected to each other in a data-communicable manner.

The biological data acquisition unit 11 acquires biological data of the user. The biological data acquisition unit 11 is also referred to as a biological data acquisition means. The “biological data” is data on various biological information generated from the inside, the surface, or the like of a living body (user). The biological information may include, but is not limited to, pulse, heart rate, blood pressure, respiration, body temperature, electroencephalogram, electrocardiogram, electromyogram. The biological data acquisition unit 11 acquires the biological data of the user from a biological sensor (not illustrated) that detects predetermined biological information. The biological sensor may be, for example, a pulse wave sensor, a heart rate sensor, a blood pressure sensor, or a respiration sensor, or may be a sensor other than these sensors. Specifically, the biological sensor may be, for example, any of a pressure sensor, a photoelectric sensor, a temperature sensor, and a visible light camera. The biological sensor may be a contact type or a non-contact type. The number of biological sensors may be one or two or more. That is, the biological data acquisition unit 11 may acquire one type of biological data or may acquire a plurality of types of biological data.

The motion data acquisition unit 12 acquires motion data indicating the motion of the user. The motion data acquisition unit 12 is also referred to as a motion data acquisition means. The “motion of the user” refers to a physical motion of the user. That is, the motion of the user refers to a part or all of the motion of the user's body. The motion of the user refers to, for example, motions for the user's head, body, upper limb, lower limb, other user's body, and a combination thereof. The motion data is data about motion information indicating the motion of the user. The motion information may be information regarding speed, acceleration, inclination, parallel movement, rotational movement, angular velocity, angle, and the like associated with the motion of the user, or may be information other than these. Specifically, the motion data may be acceleration data associated with the motion of the user, angular velocity data, or image data. The motion data acquisition unit 12 acquires motion data from a motion sensor (not illustrated) that detects motion information of the user. The motion sensor may be, for example, an acceleration sensor, a gyro sensor (angular velocity sensor), a camera, or a sensor other than these sensors. The motion sensor may be a contact type or a non-contact type. Furthermore, the number of motion sensors may be one or two or more. That is, the motion data acquisition unit 12 may acquire one type of motion data or may acquire a plurality of types of motion data.

The environment data acquisition unit 13 acquires environment data indicating the environment in which the user is placed. The environment data acquisition unit 13 is also referred to as an environment data acquisition means. The “environment” refers to, for example, a situation around the user. The environment may refer to, for example, any or all of the weather, location, and space around the user. The environment data is data about environment information around the user. The environment information may be, for example, temperature information, humidity information, weather information, atmospheric pressure information, position information, or altitude information, or may be other information. The environment data acquisition unit 13 acquires environment data from an environment sensor (not illustrated) that detects predetermined environment information. The environment sensor may be, for example, a temperature sensor, a humidity sensor, an atmospheric pressure sensor, a global positioning system (GPS), a beacon, a visible light camera, or a sensor other than these. The number of environment sensors may be one or two or more. That is, the environment data acquisition unit 13 may acquire one type of environment data or may acquire a plurality of types of environment data. The environment data acquisition unit 13 may acquire one environment data by combining a plurality of types of environment sensors. For example, the environment data acquisition unit 13 may acquire the altitude information at the current position of the user by combining the atmospheric pressure sensor and the GPS.

The biological data acquisition unit 11 and the biological sensor, the motion data acquisition unit 12 and the motion sensor, and the environment data acquisition unit 13 and the environment sensor may be data-communicably connected via a wired or wireless network. The biological data acquisition unit 11, the motion data acquisition unit 12, and the environment data acquisition unit 13 may acquire data directly from various sensors, or may acquire data via another terminal or a communication apparatus (not illustrated).

The evaluation unit 14 evaluates the health condition of the user based on the biological data acquired by the biological data acquisition unit 11, the motion data acquired by the motion data acquisition unit 12, and the environment data acquired by the environment data acquisition unit 13. The evaluation unit 14 is also referred to as an evaluation means. The evaluation unit 14 can evaluate the health condition of the user by determining, for example, whether the user is healthy, whether the user has a suspected disease, a specific type of disease, and the like. The evaluation unit 14 can generate a comment regarding the health condition of the user.

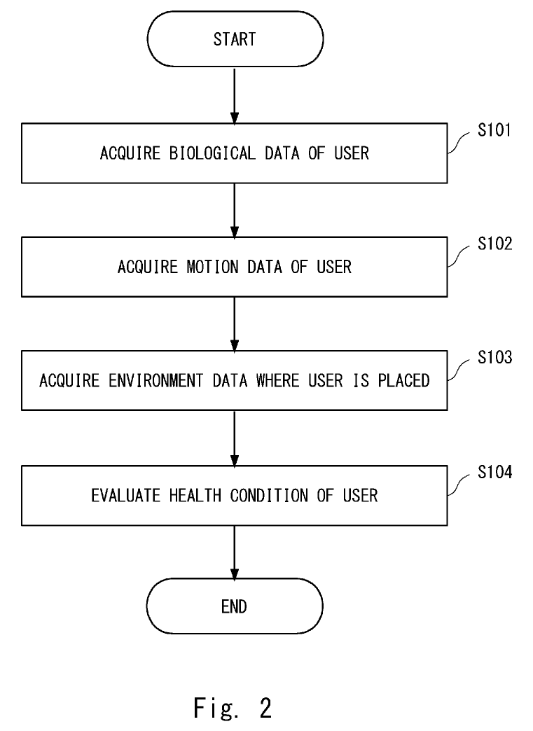

FIG. 2 is a flowchart illustrating an example of a flow of a health condition evaluation method by the health condition evaluation system 1. First, the biological data acquisition unit 11 acquires biological data of the user (S101). Next, the motion data acquisition unit 12 acquires motion data of the user (S102). Thereafter, the environment data acquisition unit 13 acquires environment data in which the user is placed (S103). Here, steps S102 and S103 may be performed before S101, or steps S101 to S103 may be performed simultaneously. Thereafter, the evaluation unit 14 evaluates the health condition of the user based on the biological data acquired by the biological data acquisition unit 11, the motion data acquired by the motion data acquisition unit 12, and the environment data acquired by the environment data acquisition unit 13 (S104).

As described above, in the first example embodiment, the health condition of the user can be evaluated based on the biological data and the motion data of the user, and the environment data indicating the environment in which the user is placed. Although the health condition of the user can be affected by various factors, the health condition evaluation system 1 can evaluate the health condition of the user in consideration of various data. Therefore, the health condition evaluation system 1 can appropriately evaluate the health condition of the user. Then, the health condition evaluation system 1 can support the decision making regarding the health condition of the user.

### Second Example Embodiment

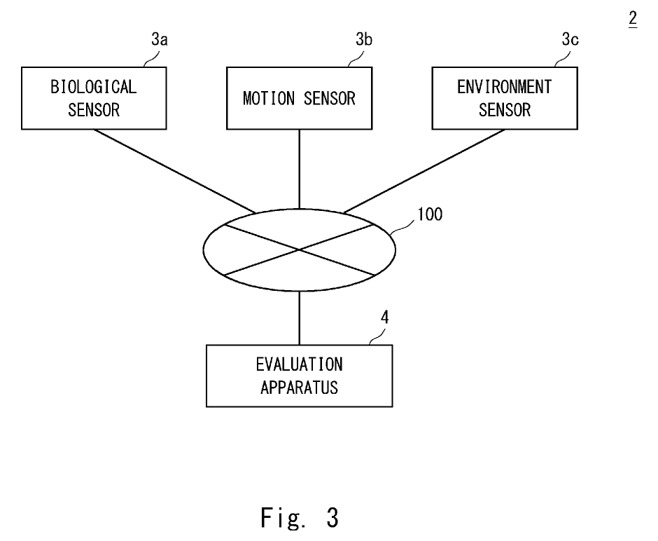

Next, a second example embodiment will be described. The second example embodiment is a specific example of the first example embodiment described above. FIG. 3 is a block diagram illustrating a configuration of a health condition evaluation system 2 according to the present disclosure. The health condition evaluation system 2 includes a biological sensor 3a, a motion sensor 3b, an environment sensor 3c, an evaluation apparatus 4, and a network 100. Hereinafter, the biological sensor 3a, the motion sensor 3b, and the environment sensor 3c may be collectively referred to as “various sensors”.

The environment sensor 3c is a sensor for detecting the environment in which the user is placed. The environment sensor 3c is, for example, a sensor for detecting the temperature, humidity, and atmospheric pressure around the user, and the position and altitude of the user. These pieces of environment information may also be referred to as “environmental parameters”. The environment sensor 3c is relevant to a sensor from which the environment data is acquired by the environment data acquisition unit 13 in the health condition evaluation system 1. The environment sensor 3c may or may not be built in a dedicated apparatus for detecting predetermined environment information. The environment sensor 3c may be built in, for example, a user terminal owned by the user. Since the description of the other environment sensor 3c overlaps with the description of the environment sensor in the health condition evaluation system 1, the description thereof will be omitted.

The biological sensor 3a is a sensor that detects biological information of the user. The biological information may include, but is not limited to, pulse, heart rate, blood pressure, respiration, body temperature, electroencephalogram, electrocardiogram, electromyogram. These pieces of biological information can also be referred to as “biological parameters”. The biological sensor 3a is relevant to a sensor from which biological data is acquired by the biological data acquisition unit 11 in the health condition evaluation system 1. The biological sensor 3a may or may not be built in a dedicated medical apparatus for detecting predetermined biological information. The biological sensor 3a may be built in, for example, a user terminal owned by the user. The user terminal is, for example, a smartphone or a wearable apparatus. The wearable apparatus is a computer apparatus that can be worn by a user. The wearable apparatus may be, for example, a smartwatch, a smart glass, a smart ring, a head-mounted display, or may be another wearable apparatus. The biological sensor 3a may be built in a camera that detects biological information of the user by imaging the user. Since the description of the other biological sensor 3a overlaps with the description of the biological sensor in the health condition evaluation system 1, the description thereof will be omitted.

The motion sensor 3b is a sensor for detecting a motion of a user. The motion sensor 3b is, for example, a sensor for detecting a speed, an inclination, a parallel movement, a rotational movement, an angle, and the like accompanying the motion of the user. These user actions may also be referred to as “motion parameters”. The motion sensor 3b is relevant to a sensor from which motion data is acquired by the motion data acquisition unit 12 in the health condition evaluation system 1. The motion sensor 3b may or may not be built in a dedicated apparatus for detecting a predetermined motion of the user. The motion sensor 3b may be built in, for example, a user terminal owned by the user. The motion sensor 3b may be built in a camera that detects the motion of the user by imaging the user. Since the description of the other motion sensor 3b overlaps with the description of the motion sensor in the health condition evaluation system 1, the description thereof will be omitted.

Each of the various sensors may be one or a plurality of sensors. That is, each of the various sensors may detect one type of information or may detect a plurality of types of information. The various sensors may be built in the same apparatus or terminal. For example, the biological sensor 3a, the motion sensor 3b, and the environment sensor 3c may be built in a wearable apparatus worn by the user. The various sensors may periodically transmit sensor data (biological data, motion data, and environment data) including detection information to the evaluation apparatus 4 at a predetermined interval, may transmit sensor data based on an instruction from the user or the evaluation apparatus 4, or may transmit both sensor data. The various sensors may transmit sensor data including all information of the detected information to the evaluation apparatus 4, or may transmit sensor data for some information to the evaluation apparatus 4. The various sensors can transmit, as sensor data (biological data, motion data, and environment data), detected information and detected time information to the evaluation apparatus 4 in association with each other. The various sensors may directly transmit detected information to the evaluation apparatus 4, or may transmit detected information to the evaluation apparatus 4 via another terminal or a communication apparatus (not illustrated).

The biological sensor 3a, the motion sensor 3b, and the environment sensor 3c are connected to the evaluation apparatus 4 via the network 100. The network 100 is a wired or wireless communication line, for example, a local area network (LAN), the Internet, or the like.

Next, the evaluation apparatus 4 will be described. The evaluation apparatus 4 is an information processing apparatus that evaluates the health condition of the user. That is, the evaluation apparatus 4 is relevant to the health condition evaluation system 1 in the first example embodiment. The evaluation apparatus 4 is typically one or more computer apparatuses that operate in a case where a processor executes a program stored in a memory. In other words, the evaluation apparatus 4 may include one computer apparatus or two or more computer apparatuses. For example, the evaluation apparatus 4 is one or more server apparatuses.

The evaluation apparatus 4 receives information detected by various sensors from the biological sensor 3a, the motion sensor 3b, and the environment sensor 3c via the network 100. The evaluation apparatus 4 can instruct various sensors to transmit detected information via the network 100.

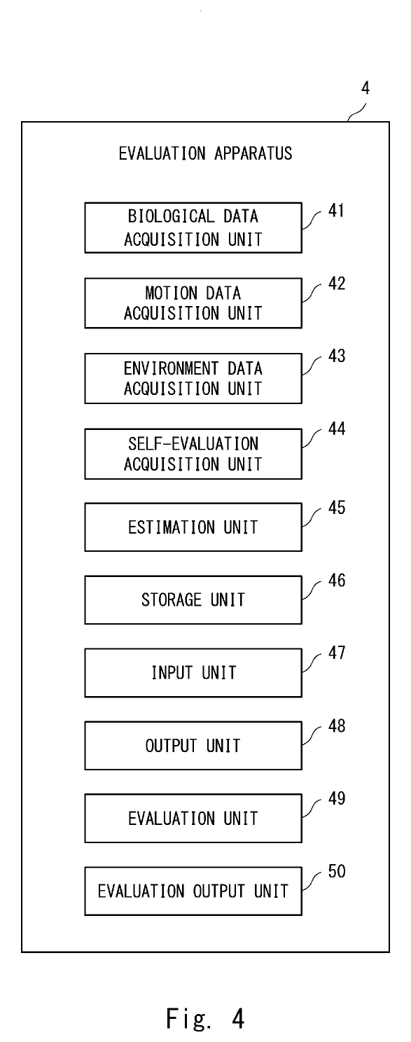

A configuration of the evaluation apparatus 4 will be described with reference to the drawings. FIG. 4 is a block diagram illustrating a configuration of an evaluation apparatus 4. The evaluation apparatus 4 includes a biological data acquisition unit 41, a motion data acquisition unit 42, an environment data acquisition unit 43, a self-evaluation acquisition unit 44, an estimation unit 45, a storage unit 46, an input unit 47, an output unit 48, an evaluation unit 49, and an evaluation output unit 50. These elements are typically software or modules in which processing is executed by the processor executing a program stored in the memory. These elements in the evaluation apparatus 4 may be hardware such as a circuit or a chip. These elements may be configured by the same computer apparatus or may be configured by different computer apparatuses.

The elements in the evaluation apparatus 4 are connected so as to be able to communicate data with each other as necessary. The data communication may be performed via a wired or wireless network. The data communication may be performed in the same computer apparatus, may be performed via an Internet, or may be performed by using a near field communication technology. A type of a communication protocol in data communication is not limited.

The biological data acquisition unit 41 acquires biological data of the user. The biological data acquisition unit 41 is also referred to as a biological data acquisition means. That is, the biological data acquisition unit 41 is relevant to the biological data acquisition unit 11 in the health condition evaluation system 1. The biological data acquisition unit 41 acquires, for example, biological data including biological information detected by a biological sensor 3a built in a wearable apparatus worn by the user. The biological data acquisition unit 41 may acquire biological data in which biological information and time information at which the biological sensor 3a has detected the biological information are associated with each other. The biological data acquisition unit 41 may acquire, as the biological data, data in which the biological information acquired from the biological sensor 3a is associated with time information at which the biological information is received from the biological sensor 3a. The biological data acquisition unit 41 may periodically acquire the biological data at a predetermined interval. The biological data acquisition unit 41 may acquire the biological data based on an instruction from the user or the like. In this case, the biological data acquisition unit 41 can instruct the biological sensor 3a to transmit the biological data. The biological data acquisition unit 41 can transmit the acquired biological data to the storage unit 46.

The motion data acquisition unit 42 acquires motion data of the user. The motion data acquisition unit 42 is also referred to as a motion data acquisition means. That is, the motion data acquisition unit 42 is relevant to the motion data acquisition unit 12 in the health condition evaluation system 1. The motion data acquisition unit 42 acquires, for example, motion data including motion information of the user detected by the motion sensor 3b built in a wearable apparatus worn by the user. The motion data acquisition unit 42 may acquire motion data in which the motion information and time information at which the motion sensor 3b has detected the motion information are associated with each other. The motion data acquisition unit 42 may acquire, as motion data, data in which the motion information acquired from the motion sensor 3b is associated with time information at which the motion information is received from the motion sensor 3b. The motion data acquisition unit 42 may acquire motion data based on an instruction from a user or the like. In this case, the motion data acquisition unit 42 can instruct the motion sensor 3b to transmit the motion data. The motion data acquisition unit 42 can transmit the acquired motion data to the storage unit 46.

The environment data acquisition unit 43 acquires environment data regarding the user. The environment data acquisition unit 43 is also referred to as an environment data acquisition means. That is, the environment data acquisition unit 43 is relevant to the environment data acquisition unit 13 in the health condition evaluation system 1. The environment data acquisition unit 43 acquires, for example, environment data including environment information detected by the environment sensor 3c built in a wearable apparatus worn by the user. The environment data acquisition unit 43 may acquire environment data in which environment information and time information at which the environment sensor 3c has detected the environment information are associated with each other. The environment data acquisition unit 43 may acquire, as the environment data, data in which the environment information acquired from the environment sensor 3c is associated with time information at which the environment information is received from the environment sensor 3c. The environment data acquisition unit 43 may acquire the environment data based on an instruction from a user or the like. In this case, the environment data acquisition unit 43 can instruct the environment sensor 3c to transmit the environment data. The environment data acquisition unit 43 can transmit the acquired environment data to the storage unit 46.

The environment data acquisition unit 43 can acquire prediction environment data indicating a prediction environment in which the user is to be placed in the future. That is, the environment data acquisition unit 43 can acquire the prediction environment data as at least a part of the environment data. The prediction environment may include, for example, an environment 5 minutes ahead, 1 hour ahead, 12 hours ahead, 1 day ahead, or 1 week ahead, or may include other future environments. The environment data acquisition unit 43 may acquire the prediction environment data, for example, by performing data communication with a predetermined environment prediction system (not illustrated) that generates prediction environment data regarding the environment in which the user is placed. The prediction environment data indicates data in which the prediction environment information and the time information are associated with each other. The environment prediction system may be, for example, a weather forecasting system.

The environment data acquisition unit 43 may generate and acquire the prediction environment data by analyzing the current or past motion data of the user acquired by the motion data acquisition unit 42 and the current or past environment data acquired by the environment data acquisition unit 43. For example, the environment data acquisition unit 43 can predict the position information and the altitude information of the user at a predetermined timing by analyzing the current or past motion data acquired by the motion data acquisition unit 42 and the current or past environment data acquired by the environment data acquisition unit 43. The environment data acquisition unit 43 may acquire the prediction environment data by, for example, a trained model (not illustrated) learned using a predetermined machine learning algorithm. In this case, in the learning model, parameters and the like are learned using training data in which user's motion data and environment data are input and prediction environment data is output. As the machine learning algorithm, for example, a Recurrent Neural Network (RNN) or the like can be used. The environment data acquisition unit 43 inputs the motion data acquired by the motion data acquisition unit 42 and the environment data acquired by the environment data acquisition unit 43 to the trained model. Then, the environment data acquisition unit 43 outputs the prediction environment data from the trained model. As a result, the environment data acquisition unit 43 can acquire the prediction environment data.

The environment data acquisition unit 43 may acquire the prediction environment data using both the prediction result by the environment prediction system and the analysis result of the motion data and the environment data. For example, the environment data acquisition unit 43 can predict the position information of the user at a predetermined timing by analyzing the motion data of the user, and can acquire the weather information of the position at the timing from the weather prediction system as the prediction environment data. The environment data acquisition unit 43 can transmit the acquired prediction environment data to the storage unit 46.

The self-evaluation acquisition unit 44 acquires a self-evaluation on the health condition of the user. The self-evaluation acquisition unit 44 is also referred to as a self-evaluation acquisition means. The “self-evaluation” is a subjective evaluation on the health condition of the user. The self-evaluation includes the user's own health degree and the user's subjective symptom. The “health degree” means a physical or mental health degree. The health degree is, for example, contents such as “I feel good”, “I feel bad”, “I feel healthy”, and “I feel unhealthy”. The “subjective symptom” refers to a physical or mental abnormality or change that the user can feel. The subjective symptom may refer to, for example, any of “headache”, “dizziness”, “nausea”, “shortness of breath”, “abdominal pain”, and “depressed mood”, or may refer to other subjective symptoms. Typically, the self-evaluation acquisition unit 44 acquires the self-evaluation on the current health condition of the user, but may acquire the self-evaluation on the past health condition. The self-evaluation acquisition unit 44 can receive user's self-evaluation for a plurality of evaluation items. The self-evaluation acquisition unit 44 can transmit the self-evaluation data in which the acquired self-evaluation of the user and the acquisition time information are associated to the storage unit 46.

The self-evaluation acquisition unit 44 may periodically acquire the self-evaluation of the user, may acquire the self-evaluation based on intention of the user, or may acquire the self-evaluation by the self-evaluation acquisition unit 44 instructing the user at a predetermined timing. The self-evaluation acquisition unit 44 can acquire the self-evaluation of the user by receiving an input of the user’s self-evaluation from the user via a predetermined input apparatus (not illustrated). The input apparatus may be a microphone, a touch panel, a mouse, or a keyboard, or may be another input apparatus. The input apparatus may be built in a user terminal owned by the user. For example, the input apparatus may be built in a smartphone or a wearable apparatus owned by the user. That is, the input apparatus may be built in the same user terminal as the biological sensor 3a, the motion sensor 3b, and the environment sensor 3c.

The self-evaluation may be numerical information, character string information, or both. That is, at least a part of the self-evaluation may be numerical information or character string information. In the case of numerical information, the user may select self-evaluation from a predetermined category via the input apparatus, for example. A plurality of categories may be set for each evaluation item. For example, categories such as “5: I feel very good”, “4: I feel generally good”, “3: neither good nor bad”, “2: I feel not very good”, and “1: I feel not good” may be set for the “health degree”. In this case, the self-evaluation acquisition unit 44 can output the choices of the category to the user via a predetermined output apparatus (not illustrated). The output apparatus may be, for example, a speaker or a display, or may be another output apparatus. The output apparatus may be built in a user terminal such as a smartphone or a wearable apparatus owned by the user. That is, the output apparatus may be built in the same user terminal as the various sensors. The user can input the self-evaluation on the health degree from the category into the input apparatus. Then, the self-evaluation acquisition unit 44 can acquire numerical information of the user's self-evaluation on the health degree.

In the case of the character string information, the self-evaluation acquisition unit 44 can output, for example, content prompting the user to input the character string information regarding the self-evaluation via the output apparatus. For example, the self-evaluation acquisition unit 44 can output “Enter your self-evaluation.” to the output apparatus. Then, the user can input the self-evaluation by a character string via the input apparatus. The character string may be a word or a sentence. The number of sentences may be one or plural. For example, the user may input “I feel good” for the user's health degree, or may input “I have a headache” for the subjective symptom. The user may input self-evaluation over a plurality of evaluation items by one sentence. For example, the user may input “I feel bad because I have a headache”. In this manner, the self-evaluation acquisition unit 44 can acquire the character string information of the self-evaluation of the user.

In the case of the character string information, the self-evaluation acquisition unit 44 may classify the self-evaluation of the user into a predetermined category by analyzing the self-evaluation input by the user. As a result, the self-evaluation acquisition unit 44 may convert the self-evaluation input by the user as the character string information into numerical information. The category of the self-evaluation may be classified according to the health degree of the user, may be classified according to the subjective symptom of the user, or may be classified according to both of them. The self-evaluation acquisition unit 44 can classify the self-evaluation of the user into a predetermined category by, for example, a trained model (not illustrated) learned using a predetermined machine learning algorithm. In this case, in the trained model, parameters and the like are learned by using training data in which character string information of self-evaluation regarding the health condition of the user is input and a category of the self-evaluation is output. The machine learning algorithm may be an RNN. The self-evaluation acquisition unit 44 may classify the self-evaluation of the user by inputting the self-evaluation of the user to the trained model and outputting a category of the self-evaluation.

The estimation unit 45 estimates the activity state of the user based on at least the motion data acquired by the motion data acquisition unit 42. The estimation unit 45 is also referred to as an estimation means. “Activity state” refers to a state of physical activity by a user. The activity state may refer to an action of the user such as “walking”, “running”, “stair climbing”, “bicycle”, or “jump”. The activity state may refer to a specific workout such as “walking”, “running”, “cycling”, or “swimming”. Further, the activity state may refer to sports such as “baseball”, “soccer”, “basketball”, “golf”, and “trail running”. Furthermore, the estimation unit 45 may estimate the number of steps, the moving distance, the instantaneous or average speed in a predetermined period as the activity state. The estimation unit 45 may estimate that the user is in a sitting posture, resting, sleeping, or the like, as the activity state of the user. That is, the estimation unit 45 may estimate that the user is not performing a continuous motion as the activity state.

The estimation unit 45 may estimate the activity state of the user based on not only the current motion data acquired by the motion data acquisition unit 42 but also the past motion data. The estimation unit 45 may estimate the activity state of the user based on the current or past biological data acquired by the biological data acquisition unit 41 and the current or past environment data acquired by the environment data acquisition unit 43. The current or past sensor data acquired by the biological data acquisition unit 41, the motion data acquisition unit 42, and the environment data acquisition unit 43 is stored in the storage unit 46. For example, the estimation unit 45 can estimate that the user is walking based on the current or past motion information and position information of the user. The estimation unit 45 can estimate that the user is trail running by using the current or past motion information, position information, and altitude information of the user.

The estimation unit 45 can estimate the activity state of the user by analyzing predetermined sensor data. The estimation unit 45 can estimate the activity state of the user by, for example, a trained model (not illustrated) learned using a predetermined machine learning algorithm. In this case, in the trained model, parameters and the like are learned by using training data in which at least motion data of the user is input and an activity state of the user is output. Here, the learning model may use training data using biological data or environment data of the user as an input. The machine learning algorithm may be an RNN. The estimation unit 45 inputs at least the motion data acquired by the motion data acquisition unit 42 to the trained model. Then, the estimation unit 45 outputs the activity state of the user with respect to the input motion data from the trained model. Thus, the estimation unit 45 can estimate the activity state as the activity state of the user. In a case where the trained model uses the biological data and the environment data of the user as the training data, the estimation unit 45 can input the biological data acquired by the biological data acquisition unit 41 and the environment data acquired by the environment data acquisition unit 43 to the trained model together with the motion data. The estimation unit 45 may input current or past sensor data to the trained model.

The estimation unit 45 can transmit the estimated activity state to the storage unit 46. Specifically, the estimation unit 45 can transmit the activity state data in which the estimated activity state and the time information are associated with each other to the storage unit 46. The activity state data may be, for example, data indicating an action of the user at a predetermined timing, data indicating contents of a workout at a predetermined timing, or data indicating contents of a sport at a predetermined timing. The activity state data may be data indicating an estimated number of steps, an estimated moving distance, and an estimated speed in a predetermined period based on time information. The estimation unit 45 may transmit one piece of activity state data to the storage unit 46, or may transmit a plurality of pieces of activity state data to the storage unit 46.

The storage unit 46 includes, for example, a storage apparatus such as a hard disk or a flash memory. The storage unit 46 may have a function of connecting to a storage apparatus or a storage system such as a cloud storage, a network attached storage (NAS), or a storage area network (SAN) via a predetermined network. The storage unit 46 is also referred to as a storage means. The storage unit 46 stores evaluation criteria for evaluating the health condition of the user and various data.

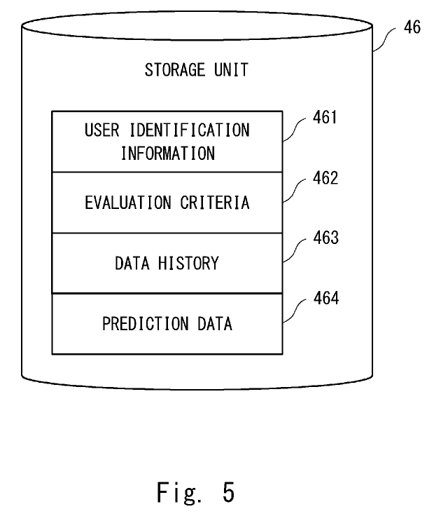

Here, contents stored in the storage unit 46 will be described with reference to the drawings. FIG. 5 is a block diagram illustrating contents stored in the storage unit 46. The storage unit 46 can store user identification information 461, evaluation criteria 462, data history 463, and prediction data 464. The storage unit 46 stores the user identification information 461, the evaluation criteria 462, the data history 463, and the prediction data 464 in association with each other. The user identification information 461 is identification information of the user. The user identification information 461 may be, for example, an identification (ID) of the user, a name of the user, or both. The evaluation criteria 462 are criteria that can be used for evaluating the health condition of the user. Specifically, the evaluation criteria 462 are criteria that can be used to determine whether the user is healthy, whether the user has a suspected disease, a specific type of disease, and the like. The evaluation criteria 462 can be determined mainly by the environment data acquired by the environment data acquisition unit 43 and the activity state of the user estimated by the estimation unit 45. In other words, the evaluation criteria 462 can be determined based on the environment in which the user is placed and the activity state of the user. For example, the evaluation criteria 462 are determined for each environmental parameter and each activity state. Specifically, the evaluation criteria 462 include a reference value of each biological parameter relevant to the environmental parameter and the activity state. Here, the “reference value” is a value or a range used for determining whether the health condition is good or bad. That is, the evaluation criteria 462 describe, for example, a correspondence relationship between the environmental parameter, and the activity state of the user and the reference value of the biological data.

For example, in a case where the predetermined biological parameter falls within the reference value, it is determined that the health condition of the user for the biological parameter is good. In a case where the plurality of biological parameters is within the reference value, it is determined that the health condition of the user for the plurality of biological parameters is good. The evaluation criteria 462 may include a criterion that the overall health condition of the user is “good” in a case where a parameter of a predetermined value or more among the plurality of biological parameters falls within the reference value. Similarly, the evaluation criteria 462 may include a criterion that the overall health condition of the user is “poor” in a case where a parameter less than a predetermined value among the plurality of biological parameters falls within the reference value. The evaluation criteria 462 may include a criterion for classifying the overall health condition of the user into a plurality of categories, for example, according to the number of biological parameters within the reference value. The evaluation criteria 462 may include a criterion for determining a disease suspected of the user based on the value of the biological parameter. For example, the evaluation criteria 462 may include a criterion of whether the user has heat stroke. In this case, the evaluation criteria 462 may include reference values of the user's body temperature, pulse, and blood pressure in a case where heat stroke is suspected.

The evaluation criteria 462 can include feedback on the activity state of the user determined based on the health condition of the user. The “feedback on the activity state” includes, for example, suggestion of the presence or absence of a health problem of the user with respect to the activity state and change of the activity state. Specifically, the feedback on the activity state can include that the activity is dangerous in terms of health, that the activity should be stopped, that the pace of the activity should be changed, and the like. The evaluation criteria 462 may include feedback for each biological parameter. For example, in a case where the heart rate of the user who runs in the scorching heat is higher than the reference value, the evaluation criteria 462 may include feedback that the running should be stopped because there is a risk of heat stroke. The evaluation criteria 462 may include feedback indicating that the pace of running should be reduced. Alternatively, the evaluation criteria 462 may include feedback indicating that the running should be stopped in a case where a plurality of biological parameters such as the body temperature, the heart rate, and the blood pressure of the user who runs in the scorching heat deviate from the reference value.

The evaluation criteria 462 may be determined by self-evaluation on the health condition of the user acquired by the self-evaluation acquisition unit 44. In other words, the evaluation criteria 462 may be determined based on self-evaluation on the health condition of the user. That is, the evaluation criteria 462 may describe a correspondence relationship between the self-evaluation on the health condition of the user and the reference value of the biological data. The evaluation criteria 462 may use the self-evaluation of the user as supplementary information, for example, in evaluating the health condition of the user. In a case where the self-evaluation input by the user via the input apparatus is numerical information, the evaluation criteria 462 may be determined for each numerical value. In a case where the self-evaluation input by the user is character string information, the evaluation criteria 462 may be determined for each category of the self-evaluation acquired by the self-evaluation acquisition unit 44 analyzing the self-evaluation. In a case where the evaluation criteria 462 describes a correspondence relationship between the self-evaluation on the health condition of the user and the reference value of the biological data, the evaluation criteria 462 may be determined based on a correspondence relationship between the past self-evaluation of the user and the biological data of the user at the timing. That is, the evaluation criteria 462 may be determined based on history information in which user's self-evaluation and biological data are associated with each other.

The data history 463 is history information including the current or past biological data acquired by the biological data acquisition unit 41, the current or past motion data acquired by the motion data acquisition unit 42, and the current or past environment data acquired by the environment data acquisition unit 43. In the data history 463, information acquired by each acquisition unit and time information are associated with each other. The data history 463 may include the current or past self-evaluation on the health condition of the user acquired by the self-evaluation acquisition unit 44. In this case, in the data history 463, the self-evaluation and the time information at which the self-evaluation is acquired by the self-evaluation acquisition unit 44 are further associated with each other. The prediction data 464 includes the prediction environment data acquired by the environment data acquisition unit 43. In the prediction data 464, for example, time information at which prediction is performed, time information at a prediction timing, and predicted prediction environment information are associated with each other. The data history 463 is history information that can be used to define the evaluation criteria 462.

Returning to FIG. 4, the description of the configuration of the evaluation apparatus 4 will be continued. The input unit 47 inputs the biological data acquired by the biological data acquisition unit 41, the motion data acquired by the motion data acquisition unit 42, and the environment data acquired by the environment data acquisition unit 43 to a predetermined trained model (not illustrated). For example, the input unit 47 inputs the current or past biological data, motion data, or environment data acquired by each acquisition unit, stored in the storage unit 46, to a predetermined trained model. The input unit 47 may input the prediction environment data stored in the storage unit 46 to a predetermined trained model. The input unit 47 is also referred to as an input means.

The trained model to which the input unit 47 inputs the sensor data and the like can output the evaluation of the health condition of the user in a case where the biological data, the motion data, and the environment data of the user are input. The “evaluation of the health condition” is, for example, whether the user is healthy, whether the user has a suspected disease, a specific type of disease, a comment on the health condition of the user, and the like. The trained model may output feedback on the activity state of the user estimated based on at least the motion data of the user. That is, the trained model may output a suggestion about the presence or absence of a health problem of the user with respect to the estimated activity state and the activity state. In this case, the trained model may output the estimated activity state of the user together. The data output by the trained model is typically data in a text format.

The trained model can be generated by using a machine learning algorithm such as RNN. That is, the learning model is learned using a machine learning algorithm. In the case of RNN, the trained model includes an input layer, an output layer, and an intermediate layer. The biological data of the user, the motion data, and the environment data are input to the input layer. The output layer outputs the evaluation of the health condition of the user with respect to the input biological data, motion data, and environment data of the user. In the intermediate layer, parameters are learned using training data in which biological data, motion data, and environment data of the user are input and evaluation of the health condition of the user is output. The trained model generated in this manner can cause the computer to function to input the biological data, the motion data, and the environment data of the user to the input layer, perform calculation in the intermediate layer, and output the evaluation of the health condition of the user from the output layer. The trained model may be generated for each user or may be generated for each of a plurality of users. The trained model may further use the prediction environment data as training data. In a case where the trained model outputs feedback for the activity state of the user, the learning model may use at least the activity state of the user relevant to the motion data of the user as the training data.

The input unit 47 can further input the self-evaluation on the health condition of the user to the trained model. In this case, the trained model can output the evaluation of the health condition for the self-evaluation of the user input by the input unit 47. In this case, in the trained model, the parameter is learned using training data in which the self-evaluation of the user is input as a part of the training data and the evaluation of the health condition of the user is output.

In a case where the input unit 47 further inputs the prediction environment data, the trained model can output the evaluation of the future health condition of the user. The evaluation of the future health condition may be, for example, the evaluation of the health condition of the user at a prediction timing in the prediction environment data, or may be the evaluation of the health condition of the user at a certain timing in another future. In this case, the trained model may output feedback on the activity state of the user based on the future health condition. For example, in a case where the future health condition of the user is predicted to be “poor”, the trained model may output feedback indicating that the activity state of the user is dangerous in terms of health, and thus the activity should be stopped.

The trained model may output instruction information indicating an instruction to input further biological data, motion data, or environment data. For example, in a case where the biological data, the motion data, or the environment data is insufficient in outputting the evaluation of the health condition, the trained model may output information indicating the insufficient data. For example, in a case where the trained model determines that the user is outdoors from the sensor data input to the trained model and the weather data is insufficient in the evaluation of the health condition of the user, the trained model may output information indicating that the weather data is insufficient.

The output unit 48 can output the evaluation of the health condition of the user for the biological data, the state data, and the environment data of the user input by the input unit 47 from the trained model. The output unit 48 is also referred to as an output means. That is, the output unit 48 can output, from the trained model, for example, whether the user is healthy, the presence or absence of a disease suspected of the user, a specific disease type, a comment on the health condition of the user, and the like. The output unit 48 can output feedback on the activity state of the user from the trained model.

Furthermore, the output unit 48 can output, from the trained model, instruction information indicating an instruction to input further biological data, motion data, or environment data. In this case, for example, by analyzing the instruction information, the output unit 48 can instruct the biological data acquisition unit 41, the motion data acquisition unit 42, or the environment data acquisition unit 43 to acquire the biological data, the motion data, or the environment data required by the trained model. Here, the output unit 48 may instruct the environment data acquisition unit 43 to acquire the prediction environment data. The biological data acquisition unit 41, the motion data acquisition unit 42, or the environment data acquisition unit 43 acquires necessary data based on an instruction from the output unit 48. Then, the biological data acquisition unit 41, the motion data acquisition unit 42, or the environment data acquisition unit 43 transmits the acquired data to the storage unit 46. The input unit 47 can further input the biological data acquired by the biological data acquisition unit 41 in this manner to the trained model. As a result, the output unit 48 can output the evaluation of the health condition of the user and the feedback on the activity state of the user from the trained model.

The evaluation unit 49 evaluates the health condition of the user based on the biological data acquired by the biological data acquisition unit 41, the motion data acquired by the motion data acquisition unit 42, and the environment data acquired by the environment data acquisition unit 43. The evaluation unit 49 is also referred to as an evaluation means. That is, the evaluation unit 49 is relevant to the evaluation unit 14 in the health condition evaluation system 1. The evaluation unit 49 may evaluate the health condition of the user based on the evaluation criteria 462 stored in the storage unit 46, may evaluate the health condition of the user using a trained model for evaluating the health condition of the user, or may evaluate the health condition of the user using both methods.

First, the evaluation unit 49 can evaluate the health condition of the user based on the evaluation criteria 462 stored in the storage unit 46. The evaluation unit 49 can evaluate the health condition of the user based on, for example, the evaluation criteria 462 defined by the environment data acquired by the environment data acquisition unit 43 and the activity state of the user estimated by the estimation unit 45. Specifically, the evaluation unit 49 refers to the evaluation criteria 462 to acquire the reference value of the biological data of the user for the environment data acquired by the environment data acquisition unit 43 and the activity state of the user estimated by the estimation unit 45. Then, the evaluation unit 49 evaluates the health condition of the user by determining whether each biological data acquired by the biological data acquisition unit 41 is within the reference value. The evaluation unit 49 determines, for example, whether the user's health condition is good or poor based on the evaluation criteria 462. The evaluation unit 49 can determine a disease suspected of the user based on the evaluation criteria 462. Furthermore, the evaluation unit 49 can perform feedback on the health condition of the user based on the health condition of the user based on the evaluation criteria 462. For example, the evaluation unit 49 can determine that the activity state by the user is dangerous in terms of health, that the activity should be stopped, that the pace of the activity should be changed, and the like.

The evaluation unit 49 can perform feedback on the activity state of the user based on the evaluation criteria 462. That is, the evaluation unit 49 can propose the presence or absence of a health problem of the user with respect to the activity state and a change in the activity state. Here, “the evaluation unit 49 performs feedback” means that the evaluation unit 49 generates the content of the feedback. The evaluation unit 49 can perform feedback that the activity performed by the user is dangerous in terms of health, that the activity should be stopped, that the pace of the activity should be changed, and the like.

Furthermore, the evaluation unit 49 can predict the future health condition of the user in a prediction environment in which the user is placed in the future based on the evaluation criteria 462. In other words, the evaluation unit 49 can evaluate the future health condition in the prediction environment in which the user is placed based on the evaluation criteria 462. Here, the prediction environment is based on the prediction environment data acquired by the environment data acquisition unit 43. That is, the evaluation unit 49 can predict the future health condition of the user by comparing the reference value of the biological data relevant to the prediction environment data acquired by the environment data acquisition unit 43 with the biological data acquired by the biological data acquisition unit 41 by referring to the evaluation criteria 462.

Furthermore, the evaluation unit 49 can perform feedback on the activity state of the user based on the future health condition of the user in the prediction environment based on the evaluation criteria 462. That is, the evaluation unit 49 can perform feedback on the current activity state of the user estimated by the estimation unit 45 based on the predicted future health condition of the user. For example, it is assumed that the environment data acquisition unit 43 acquires position information and temperature information of the user as prediction environment data at a predetermined timing (future). It is assumed that the activity state of the user is estimated to be running. In this case, in a case where the health condition of the user at the temperature around the user position at the predetermined timing is predicted to deteriorate, the evaluation unit 49 can perform the feedback that the user should stop running. Alternatively, the evaluation unit 49 can perform feedback that the user should slow down the pace of running.

Furthermore, the evaluation unit 49 can evaluate the health condition of the user based on the self-evaluation on the health condition of the user by referring to the evaluation criteria 462. For example, the evaluation unit 49 can evaluate the health condition of the user based on the evaluation criteria 462 that describe the correspondence relationship of the reference value of the biological data with respect to the self-evaluation of the user in the predetermined activity state of the user. Specifically, the evaluation unit 49 can evaluate the health condition of the user based on the subjective symptom of the user. The evaluation unit 49 may be determined based on a correspondence relationship between the past self-evaluation of the user and the biological data of the user at the timing. Here, such a correspondence relationship may be determined based on the data history 463 stored in the storage unit 46. That is, the evaluation unit 49 may evaluate the health condition of the user based on history information in which the self-evaluation of the user and the biological data are associated with each other stored as the data history 463.

In a case where the health condition of the user is evaluated based on the evaluation criteria 462, the evaluation unit 49 can instruct the biological data acquisition unit 41, the motion data acquisition unit 42, or the environment data acquisition unit 43 to acquire further biological data, motion data, or environment data. For example, in a case where the health condition of the user is to be evaluated based on the evaluation criteria 462, in a case where the predetermined data is insufficient, the evaluation unit 49 can instruct the relevant data acquisition unit to acquire the insufficient data. In this case, the evaluation unit 49 can evaluate the health condition of the user based on data further acquired from the relevant data acquisition unit.

The evaluation unit 49 can evaluate the health condition of the user using the trained model for evaluating the health condition of the user. That is, the evaluation unit 49 can evaluate the health condition of the user based on the evaluation of the health condition of the user output by the output unit 48 with respect to the biological data, the motion data, and the environment data of the user input to the trained model by the input unit 47. Typically, the evaluation unit 49 evaluates the evaluation output by the output unit 48 by the evaluation unit 49. Similarly to the case of referring to the evaluation criteria 462, the evaluation unit 49 can perform feedback on the activity state of the user by using the trained model. The evaluation unit 49 can predict the future health condition of the user in the prediction environment by using the trained model. Furthermore, the evaluation unit 49 can perform feedback on the activity state of the user with respect to the future health condition of the user by using the trained model. Furthermore, the evaluation unit 49 can evaluate the self-evaluation of the user, for example, the health condition of the user based on the subjective symptom of the user, by using the trained model.

The evaluation output unit 50 outputs the evaluation of the health condition of the user by the evaluation unit 49. The evaluation output unit 50 is also referred to as an evaluation output means. Specifically, the evaluation output unit 50 can output evaluation of the health condition of the user by the evaluation unit 49, feedback on the activity state of the user, prediction of the future health condition of the user, feedback on the activity state of the user based on the future health condition, and the like. The evaluation output unit 50 may output evaluation or the like to a user or a medical worker such as a doctor by a predetermined output apparatus (not illustrated), or may output the evaluation or the like to another computer apparatus. The output apparatus is, for example, a display apparatus or a voice output apparatus. The display apparatus includes a display including, for example, a liquid crystal panel or organic electroluminescence in order to display the evaluation contents and the like to the user. The voice output apparatus may be a speaker for voice-outputting the evaluation contents and the like to the user. For example, the evaluation output unit 50 can output the evaluation contents and the like to the user terminal owned by the user. That is, the evaluation output unit 50 may output the evaluation content or the like to, for example, a smartphone or a wearable apparatus owned by the user. The evaluation output unit 50 may output evaluation contents and the like to the same terminal as the user terminal in which the biological sensor 3a, the motion sensor 3b, and the environment sensor 3c are built.

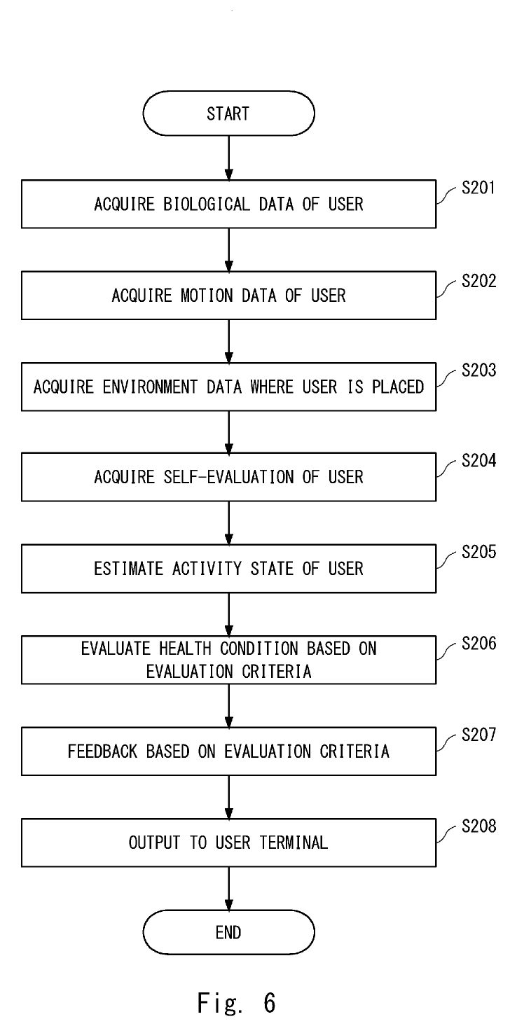

Next, a health condition evaluation method by the health condition evaluation system 2 will be described with reference to the drawings. FIG. 6 is a flowchart illustrating an example of a health condition evaluation method by the health condition evaluation system 2. Specifically, FIG. 6 illustrates an example of a case where the evaluation unit 49 evaluates the health condition of the user based on the evaluation criteria 462 stored in the storage unit 46. First, the biological data acquisition unit 41 acquires the biological data of the user (S201). Next, the motion data acquisition unit 42 acquires motion data of the user (S202). Thereafter, the environment data acquisition unit 43 acquires environment data in which the user is placed (S203). Thereafter, the self-evaluation acquisition unit 44 acquires a self-evaluation on the health condition of the user (S204). Here, steps S202 to S204 may be performed before S201, or steps S201 to S204 may be performed simultaneously.

Thereafter, the estimation unit 45 estimates the activity state of the user based on at least the motion data (S205). Thereafter, the evaluation unit 49 evaluates the health condition of the user based on the evaluation criteria 462 stored in the storage unit 46 (S206). Specifically, the evaluation unit 49 refers to the evaluation criteria 462 to determine whether the biological data of the user is within the reference value of the biological data for the acquired environment data, the self-evaluation, and the estimated activity state of the user. The evaluation unit 49 evaluates the health condition of the user based on such a determination result. Thereafter, the evaluation unit 49 performs feedback on the activity state of the user based on the evaluation criteria 462 (S207). That is, the evaluation unit 49 generates the content of feedback for the activity state of the user based on the evaluation criteria 462. Thereafter, the evaluation output unit 50 outputs the evaluation contents and the like by the evaluation unit 49 to the user terminal owned by the user (S208).

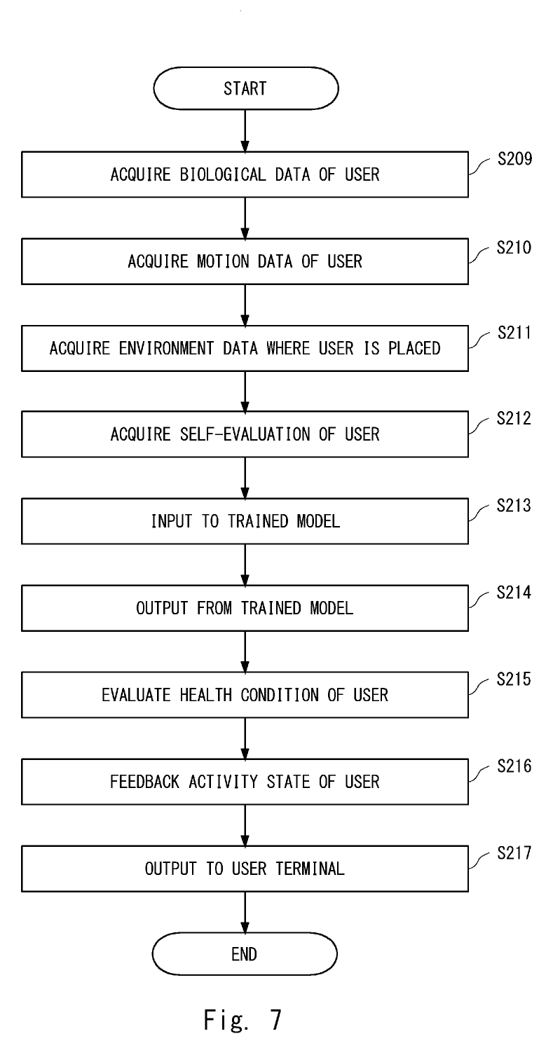

FIG. 7 is a flowchart illustrating another example of the health condition evaluation method by the health condition evaluation system 2. Specifically, FIG. 7 illustrates an example of a case where the evaluation unit 49 evaluates the health condition of the user using the trained model. Since steps S209 to S212 are the same as steps S201 to S204 in FIG. 6, the description thereof is omitted. In step S213, the input unit 47 inputs the acquired biological data, motion data, environment data, and user's self-evaluation to the trained model (S213). Next, the output unit 48 outputs the evaluation of the health condition of the user from the trained model (S214). Here, the output unit 48 can also output feedback on the activity state of the user. Thereafter, the evaluation unit 49 evaluates the health condition of the user based on the evaluation of the health condition of the user output by the output unit 48 (S215). Typically, the evaluation unit 49 evaluates the health condition of the user output by the output unit 48 by the evaluation unit 49. Thereafter, the evaluation unit 49 performs feedback on the user's activity state (S216). That is, the evaluation unit 49 generates the content of feedback on the activity state of the user. Typically, the evaluation unit 49 generates the feedback output by the output unit 48 as the content of the feedback by the evaluation unit 49. Here, step S216 may be performed before S215, and steps S215 and S216 may be performed simultaneously. Thereafter, the evaluation output unit 50 outputs the evaluation contents and the like by the evaluation unit 49 to the user terminal owned by the user (S217).

As described above, in the second example embodiment, the first example embodiment can be specifically implemented. That is, in the health condition evaluation system 2, the evaluation apparatus 4 can acquire sensor data detected by the biological sensor 3a, the motion sensor 3b, and the environment sensor 3c. Then, the evaluation apparatus 4 can evaluate the health condition of the user based on the sensor data. In recent years, wearable apparatuses such as a smart watch have been widely used. Therefore, the biological data of the user can be acquired without using a dedicated medical apparatus. On the other hand, in a case where the biological data of the user is acquired using the wearable apparatus, the wearable apparatus acquires the biological data in a case where the user is in various states. Therefore, it may not be possible to appropriately evaluate the health condition of the user based on the biological data acquired by the wearable apparatus. For example, a case where blood pressure data of a user is acquired using a wearable apparatus, and a health condition of the user is evaluated by a related evaluation system based on a blood pressure value in a resting state will be considered. In this case, the evaluation system may perform inappropriate evaluation on the health condition of the user based on the blood pressure value of the user in a case where the user is in a non-resting state (for example, an exercise state).

According to the health condition evaluation system 2, the various sensors acquire not only biological data of the user but also motion data and environment data of the user. Then, the health condition evaluation system 2 can evaluate the health condition of the user based on the sensor data by various sensors. That is, the health condition evaluation system 2 can evaluate the health condition of the user in consideration of the motion situation of the user and the environment in which the user is placed. Therefore, the health condition evaluation system 2 can appropriately evaluate the health condition of the user.

The health condition evaluation system 2 can estimate the activity state of the user based on at least the motion data of the user. Then, the health condition evaluation system 2 can evaluate the health condition of the user based on the evaluation criteria 462 determined by the environment data and the activity state of the user. That is, since the health condition evaluation system 2 can evaluate the health condition of the user based on the specific activity state of the user, it is possible to perform evaluation with high accuracy.

Furthermore, the health condition evaluation system 2 can perform feedback on the activity state of the user based on the health condition of the user. That is, the health condition evaluation system 2 can feed back the activity state of the user in accordance with the health condition of the user. As a result, the health condition evaluation system 2 can appropriately support the user's decision making regarding activities and health conditions. The health condition evaluation system 2 can maintain a good health condition of the user in a predetermined activity state.

The health condition evaluation system 2 can evaluate the health condition of the user using a predetermined trained model. The trained model can appropriately find the relationship between the sensor data by extracting the feature amount from the biological data, the motion data, and the environment data of the user. Therefore, the health condition evaluation system 2 can perform more accurate evaluation by using the trained model.

Furthermore, the health condition evaluation system 2 can predict the future health condition of the user in a prediction environment in which the user will be placed in the future. Therefore, the health condition evaluation system 2 can prevent the user's health condition from becoming poor in the future. For example, the health condition evaluation system 2 can predict the future health condition of the user based on prediction environment data including future position information of the user and weather information at the position. That is, the health condition evaluation system 2 can predict the health condition of the user based on a specific prediction environment. Therefore, the health condition evaluation system 2 can predict the future health condition of the user with higher accuracy.

Furthermore, the health condition evaluation system 2 can perform feedback on the activity state of the user based on the future health condition of the user. That is, the health condition evaluation system 2 can perform feedback on the activity state of the user based on the future health condition of the user based on the sensor data for each user. Therefore, the health condition evaluation system 2 can perform feedback according to the specific future health condition of each user. That is, the health condition evaluation system 2 can perform individual feedback rather than uniform feedback on the activity state of the user. Therefore, the health condition evaluation system 2 can further support the activity of the user.

Furthermore, the health condition evaluation system 2 can evaluate the health condition based on self-evaluation on the health condition of the user. In particular, the health condition evaluation system 2 can evaluate the health condition based on the subjective symptom of the user. In this way, by adding the self-evaluation of the user to the determination element of the evaluation of the health condition, the health condition evaluation system 2 can determine the user's disorder that cannot be determined from the motion data or the environment data. The health condition evaluation system 2 can evaluate the health condition of the user based on the data history 463 including history information in which past self-evaluation of the user and biological data are associated with each other. That is, the health condition evaluation system 2 can evaluate the health condition for the current self-evaluation of the user after grasping the biological data of the user for the past self-evaluation of the user. Therefore, the health condition evaluation system 2 can perform evaluation with higher accuracy.

Furthermore, the health condition evaluation system 2 can evaluate the health condition of the user based on the self-evaluation including the character string information. As a result, the health condition evaluation system 2 can evaluate the health condition of the user in consideration of ambiguous information that cannot be expressed as a numerical value.

### Exemplary Hardware Configuration

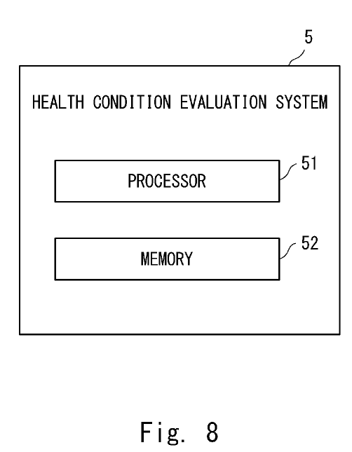

FIG. 8 is a diagram illustrating a hardware configuration example of a health condition evaluation system 5. In FIG. 8, the health condition evaluation system 5 includes a processor 51 and a memory 52. The processor 51 may be, for example, a microprocessor, a micro processing unit (MPU), or a central processing unit (CPU). The processor 51 may include a plurality of processors. The memory 52 is configured by a combination of a volatile memory and a nonvolatile memory. The memory 52 may include a storage disposed away from the processor 51. In this case, the processor 51 may access the memory 52 via an Input/Output (I/O) interface (not illustrated).

In the above-described example, a program can be stored by use of various types of non-transitory computer readable media and provided to a computer. The non-transitory computer readable media include various types of tangible storage media. Examples of the non-transitory computer readable media include a magnetic storage medium (for example, magneto-optical disk), a CD-ROM, a CD-R, a CD-R/W, and a semiconductor memory (for example, mask ROM, Programmable ROM (PROM), Erasable PROM (EPROM), flash ROM, and RAM). The program may be provided to the computer by various types of transitory computer readable media. Examples of the transitory computer readable media include electrical signals, optical signals, and electromagnetic waves. The transitory computer readable media can provide the program to the computer via a wired communication line such as an electric wire and an optical fiber or a wireless communication line. The computer includes various information processing apparatuses such as a PC, a server, a CPU, an MPU, a Field Programmable Gate Array (FPGA), and an Application Specific Integrated Circuit (ASIC).

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

Some or all of the above-described example embodiments may also be described as the following Supplementary Notes, but are not limited to the following Supplementary Notes.

### Supplementary Note 1

A health condition evaluation system including:
a biological data acquisition unit configured to acquire biological data of a user;
a motion data acquisition unit configured to acquire motion data indicating a motion of the user;
an environment data acquisition unit configured to acquire environment data indicating an environment in which the user is placed;
and an evaluation unit configured to evaluate a health condition of the user based on the biological data, the motion data, and the environment data.

### Supplementary Note 2

The health condition evaluation system according to Supplementary Note 1, further including an estimation unit configured to estimate an activity state of the user based on the motion data,
in which the evaluation unit evaluates the health condition based on evaluation criteria defined by the environment data and the activity state.

### Supplementary Note 3

The health condition evaluation system according to Supplementary Note 2, in which the evaluation unit performs feedback on the activity state of the user based on the health condition.

### Supplementary Note 4

The health condition evaluation system according to Supplementary Note 2 or 3, in which
the environment data acquisition unit acquires prediction environment data indicating a prediction environment in which the user is to be placed in a future as at least a part of the environment data, and
the evaluation unit predicts a future health condition of the user in the prediction environment.

### Supplementary Note 5

The health condition evaluation system according to Supplementary Note 4, in which the evaluation unit performs feedback on the activity state of the user based on the future health condition in the prediction environment.

### Supplementary Note 6

The health condition evaluation system according to any one of Supplementary Notes 1 to 5, further including a self-evaluation acquisition unit configured to acquire a self-evaluation on the health condition of the user,
in which the evaluation unit further evaluates the health condition based on the self-evaluation.

### Supplementary Note 7

The health condition evaluation system according to Supplementary Note 6, in which the self-evaluation acquisition unit acquires a subjective symptom of the user as the self-evaluation.

### Supplementary Note 8

The health condition evaluation system according to any one of Supplementary Notes 1 to 7, further including:
an input unit configured to input the biological data acquired by the biological data acquisition unit, the motion data acquired by the motion data acquisition unit, and the environment data acquired by the environment data acquisition unit to a trained model learned by using a machine learning algorithm, the trained model being configured to output an evaluation of a health condition of a user in a case where the biological data of the user, motion data indicating a motion of the user, and the environment data indicating an environment in which the user is placed are input; and
an output unit configured to output the evaluation of the health condition with respect to the biological data, the motion data, and the environment data input by the input unit,
in which the evaluation unit evaluates the health condition based on the evaluation output by the output unit.

### Supplementary Note 9

The health condition evaluation system according to Supplementary Note 6 or 7, further including a storage unit configured to store the self-evaluation and the biological data in association with each other as history information,
in which the evaluation unit further evaluates the health condition based on the history information.

### Supplementary Note 10

The health condition evaluation system according to any one of Supplementary Notes 1 to 9, in which the environment data includes temperature data in the environment.

### Supplementary Note 11

The health condition evaluation system according to any one of Supplementary Notes 6 to 8, in which at least a part of the self-evaluation is character string information.

### Supplementary Note 12

A health condition evaluation method for causing a computer to execute:
acquiring biological data of a user;
acquiring motion data indicating a motion of the user;
acquiring environment data indicating an environment in which the user is placed;
and evaluating a health condition of the user based on the biological data, the motion data, and the environment data.

### Supplementary Note 13

A program for causing a computer to execute:
a step of acquiring biological data of a user;
a step of acquiring motion data indicating a motion of the user;
a step of acquiring environment data indicating an environment in which the user is placed;
and a step of evaluating a health condition of the user based on the biological data, the motion data, and the environment data.

Some or all of the elements (e.g., configurations and functions) described in supplementary notes 2 to 11 dependent on supplementary note 1 can also depend on supplementary notes 12 and 13 by the dependency relationships similar to those of supplementary notes 2 to 11. Some or all of the elements described in any supplementary note can be applied to various types of hardware, software, recording means for recording software, systems, and methods.

1. A health condition evaluation system comprising:
at least one memory storing instructions, and
at least one processor configured to execute the instructions to;
acquire biological data of a user;
acquire motion data indicating a motion of the user;
acquire environment data indicating an environment in which the user is placed; and
evaluate a health condition of the user based on the biological data, the motion data, and the environment data.

2. The health condition evaluation system according to claim 1, wherein the at least one processor is further configured to execute the instructions to:
estimate an activity state of the user based on the motion data; and
evaluate the health condition based on evaluation criteria defined by the environment data and the activity state.

3. The health condition evaluation system according to claim 2, wherein the at least one processor is further configured to execute the instructions to perform feedback on the activity state of the user based on the health condition.

4. The health condition evaluation system according to claim 2, wherein the at least one processor is further configured to execute the instructions to;
acquire prediction environment data indicating a prediction environment in which the user is to be placed in a future as at least a part of the environment data; and
predict a future health condition of the user in the prediction environment.

5. The health condition evaluation system according to claim 4, wherein the at least one processor is further configured to execute the instructions to perform feedback on the activity state of the user based on the future health condition in the prediction environment.

6. The health condition evaluation system according to claim 1, wherein the at least one processor is further configured to execute the instructions to:
acquire a self-evaluation on the health condition of the user; and
evaluate the health condition further based on the self-evaluation.

7. The health condition evaluation system according to claim 6, wherein the at least one processor is further configured to execute the instructions to acquire a subjective symptom of the user as the self-evaluation.

8. The health condition evaluation system according to claim 1, wherein the at least one processor is further configured to execute the instructions to:
input the acquired biological data, the acquired motion data, and the acquired environment data to a trained model learned by using a machine learning algorithm, the trained model being configured to output an evaluation of a health condition of a user in a case where biological data of the user, motion data indicating a motion of the user, and environment data indicating an environment in which the user is placed are input;
output the evaluation of the health condition from the trained model with respect to the biological data, the motion data, and the environment data input; and
evaluate the health condition based on the evaluation output.

9. The health condition evaluation system according to claim 6, wherein the at least one processor is further configured to execute the instructions to:
store the self-evaluation and the biological data in association with each other as history information; and
evaluate the health condition further based on the history information.

10. The health condition evaluation system according to claim 1, wherein the environment data includes temperature data in the environment.

11. The health condition evaluation system according to claim 6, wherein at least a part of the self-evaluation is character string information.

12. A health condition evaluation method for causing a computer to execute:
acquiring biological data of a user;
acquiring motion data indicating a motion of the user;
acquiring environment data indicating an environment in which the user is placed; and
evaluating a health condition of the user based on the biological data, the motion data, and the environment data.

13. A non-transitory computer-readable medium storing a program for causing a computer to execute:
a step of acquiring biological data of a user;
a step of acquiring motion data indicating a motion of the user;
a step of acquiring environment data indicating an environment in which the user is placed; and
a step of evaluating a health condition of the user based on the biological data, the motion data, and the environment data.
